(19) United States
(12) Patent Application Publication
(10) Pub. No.: US 2021/0060013 A1
(43) Pub. Date: Mar. 4, 2021

(54) Method for Preventing Triple Negative Breast Cancer in Predisposed Subjects

(71) Applicant: Southern University of Science and Technology, Shenzhen (CN)

(72) Inventors: Ruijun Tian, Shenzhen (CN); Yin Sun, Shenzhen (CN); Pengfei Li, Shenzhen (CN); Bizhu Chu, Shenzhen (CN)

(21) Appl. No.: 16/560,811

(22) Filed: Sep. 4, 2019

(51) Int. Cl. A61K 31/506; A61P 35/00

(52) U.S. Cl. CPC A61K 31/506; A61K 9/006; A61P 35/00

(57) ABSTRACT

The present invention includes methods for preventing the development of triple negative breast cancer in patients predisposed to and at high risk for developing the disease comprising an effective amount of crenolanib or a salt thereof.

### CROSS-REFERENCE TO RELATED APPLICATIONS

None.

### TECHNICAL FIELD OF THE INVENTION

This invention pertains to crenolanib, or salts thereof, for prevention of the development of triple negative breast cancer in subjects predisposed to breast cancer in which an effective dose of crenolanib is administered to a subject at risk for breast cancer.

### STATEMENT OF FEDERALLY FUNDED RESEARCH

None.

### INCORPORATION-BY-REFERENCE OF MATERIALS FILED ON COMPACT DISK

None.

### BACKGROUND OF THE INVENTION

Without limiting the scope of the invention, its background is described in connection with PDGFR tyrosine kinases and their role in triple negative breast cancer development and the administration of crenolanib, or a pharmaceutically acceptable salt thereof, to subjects at risk of development of triple negative breast cancer in order to prevent occurrence of said cancer.

The platelet-derived growth factor receptors (PDGFRα and PDGFRβ) are receptor tyrosine kinases that have been shown to be directly involved in breast cancer. High expression of PDGFRβ in subjects with breast cancer is correlated to increased metastasis and a worse disease prognosis (Frings et al., 2013; Hammer et al., 2017; Heldin, Lennartsson, & Westermark, 2017; Lev et al., 2005; Paulsson, Ehnman, & Ostman, 2014). Signaling through PDGFRs plays a critical role in normal physiological process, including wound healing, inflammation, angiogenesis, and embryogenesis (Criscitiello, Gelao, Viale, Esposito, & Curigliano, 2014). During breast cancer development, this signaling pathway becomes dysregulated, and altered PDGFR signaling has been linked to aggressive cancer with a lower chance of survival (Criscitiello et al., 2014).

In breast cancer there are targeted agents available for patients whose tumors are characterized by the overexpression of the estrogen (ER) and/or progesterone (PR) hormone receptors or human epidermal growth factor receptor 2 (HER2). However, for patients whose tumors do not overexpress these markers, referred to as triple-negative breast cancer (TNBC), there are no such targeted therapies. The lack of druggable targets in this class of breast cancer has made treating this disease difficult, and patients with TNBC are at a higher risk of relapse or death (Harbeck & Gnant, 2017).

Thus, a need remains for agents that can target and prevent the development of TNBC in high-risk patients.

### SUMMARY OF THE INVENTION

In one embodiment, the present invention includes a method for preventing the development of triple negative breast cancer in a subject predisposed to breast cancer comprising administering to the subject an effective amount of crenolanib or pharmaceutically acceptable salt thereof. In one aspect, the breast cancer is not inflammatory breast cancer. In another aspect, the effective amount of crenolanib is from about 50 mg to 500 mg per day, 100 to 450 mg per day, 200 to 400 mg per day, 300 to 500 mg per day, 350 to 500 mg per day, or 400 to 500 mg per day. In another aspect, the effective amount of crenolanib is administered at least one of continuously, intermittently, systemically, or locally. In yet another aspect, the effective amount of crenolanib is administered orally, intravenously, or intraperitoneally. In another aspect, the effective amount of crenolanib is administered up to three times a day for as long as the subject is at risk for development of breast cancer. In another aspect, the crenolanib is crenolanib besylate, crenolanib phosphate, crenolanib lactate, crenolanib hydrochloride, crenolanib citrate, crenolanib acetate, crenolanib toluenesulphonate, or crenolanib succinate.

In another embodiment, the present invention includes method for preventing the development of triple negative breast cancer in a subject comprising: identifying a subject that is predisposed to a breast cancer; and administering to the subject an effective amount of crenolanib or pharmaceutically acceptable salt thereof. In one aspect, the breast cancer is not inflammatory breast cancer. In another aspect, the effective amount of crenolanib is from about 50 mg to 500 mg per day, 100 to 450 mg per day, 200 to 400 mg per day, 300 to 500 mg per day, 350 to 500 mg per day, or 400 to 500 mg per day. In another aspect, the effective amount of crenolanib is administered at least one of continuously, intermittently, systemically, or locally. In yet another aspect, the effective amount of crenolanib is administered orally, intravenously, or intraperitoneally. In another aspect, the effective amount of crenolanib is administered up to three times a day for as long as the subject is at risk for development of breast cancer. In another aspect, the crenolanib is crenolanib besylate, crenolanib phosphate, crenolanib lactate, crenolanib hydrochloride, crenolanib citrate, crenolanib acetate, crenolanib toluenesulphonate, or crenolanib succinate.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C show the ability of the present invention to prevent the development of mammary tumors in a subject genetically predisposed and at high risk of developing breast cancer. MMTV-PyMT mice were administered crenolanib starting at 8 weeks of age, before the development of palpable mammary tumors. FIG. 1A: Mice were treated with 20 mg/kg crenolanib, dissolved in vehicle, intraperitoneally once daily for 35 days. FIG. 1B: Tumor sizes were measured using digital calipers every 5 days during treatment and volumes were calculated using a standard formula ((length×width)/2)). Control mice were treated with vehicle alone. FIG. 1C: At the end of crenolanib treatment, mice were sacrificed, and final tumor weight was determined.

FIG. 2 shows the ability of the present invention to inhibit induced PDGFRβ pathway signaling activation. Conditioned media from the human TNBC cell line MDA-MB468 was applied to human mammary fibroblasts, which resulted in an increase in PDGFRβ signaling (middle lane of left half of figure). When fibroblasts were pretreated with crenolanib, it blocked this increased signaling (right lane). Similar results were seen when fibroblasts were directly treated with PDGFB, a ligand for PDGFRβ (right half of figure).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

### Definitions

As used herein, the term “subject” or “patient” are used interchangeably to refer to an animal, such as a mammal or a human, who has been the object of treatment, observation, or experiment.

As used herein, the terms “prevent”, “preventing”, “prevention” and the like refer to administering crenolanib or a pharmaceutical salt thereof to a patient or subject, prior to the onset of a disease, disorder, condition or symptom thereof, so as to prevent, suppress, inhibit or reduce, either temporarily or permanently, a subject's risk of developing breast cancer or delaying the onset thereof. In certain instances, the terms also refer to slowing the progression of breast cancer, such as inhibiting progression thereof to a harmful or otherwise undesired state.

As used herein, the term “predisposed” refers to a subject or patient that has one or more risk factors for a disease, for example, genetic or other factors (such as a family history of breast cancer) that can cause the subject to develop breast cancer. For example, a subject is predisposed to breast cancer if the one or more factors indicate the possible development of breast cancer but the subject does not yet experience or exhibit symptoms of the disease.

As used herein, the term “in need of prevention” refers to a judgment made by a physician or other caregiver that a subject or patient requires or will benefit from preventative care. This judgment is made based on a variety of factors that are in the realm of a physician's or caregiver's expertise.

As used herein, the terms “treat”, “treating”, and “treatment” refer to the administration of one or more active ingredients, compounds, salts, or compositions that prevent, reduce, or delay the onset of the symptoms or complications of breast cancer. “Treating” further refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the disease less debilitating. The treatment or amelioration of symptoms can be based on objective or subjective parameters; including the results of an examination by a physician. Accordingly, the term “treating” includes the administration of the compounds, salts, or agents of the disclosure to prevent or delay, to alleviate, or to arrest or inhibit development of the symptoms or conditions associated with breast cell proliferation, breast cancer, and breast cancer metastasis.

As used herein, the terms “proliferative disorder(s)” and “cell proliferative disorder(s)” refer to excess cell proliferation of one or more subset of cells in a multicellular organism resulting in harm (i.e. discomfort or decreased life expectancy) to the multicellular organism. Cell proliferative disorders can occur in different types of animals and humans.

As used herein, the term “triple negative breast cancer” or “TNBC” refers to a breast cancer in a subject which is not considered to overexpress hormone receptors: estrogen and/or progesterone, or HER2, irrespective of other markers or genetic alterations.

As used herein, the term “therapeutically effective amount” refers to an amount of crenolanib or a pharmaceutically acceptable salt thereof, administered to a subject as a single agent or in combination with another pharmaceutical agent(s), e.g., a chemotherapeutic agent, that in combination elicits the biological or medicinal response in a subject that is being sought by a researcher, veterinarian, medical doctor, or other clinician, which includes alleviation of the symptoms of the disease or disorder being treated. Methods for determining therapeutically effective doses for pharmaceutical compositions comprising a compound of the present invention are known in the art. Techniques and compositions for making useful dosage forms using the present invention are described in many references, including: P. O. Anderson, J. E. Knoben, and W. G. Troutman, Handbook of clinical drug data, 10th ed. New York; Toronto: McGraw-Hill Medical Pub. Division, 2002, pp. xvii, 1148 p (Anderson, Knoben, & Troutman, 2002); A. Goldstein, W. B. Pratt, and P. Taylor, Principles of drug action: the basis of pharmacology, 3rd ed. New York: Churchill Livingstone, 1990, pp. xiii, 836 p. (Goldstein, Pratt, & Taylor, 1990); B. G. Katzung, Basic & clinical pharmacology, 9th ed. (Lange medical book). New York: Lange Medical Books/McGraw Hill, 2004, pp. xiv, 1202 p. (Katzung, 2004); L. S. Goodman, J. G. Hardman, L. E. Limbird, and A. G. Gilman, Goodman and Gilman's the pharmacological basis of therapeutics, 10th ed. New York: McGraw-Hill, 2001, pp. xxvii, 2148 p. (Goodman, Hardman, Limbird, & Gilman, 2001); J. P. Remington and A. R. Gennaro, Remington: the science and practice of pharmacy, 20th ed. Baltimore, Md.: Lippincott Williams & Wilkins, 2000, pp. xv, 2077 p. (Remington & Gennaro, 2000); W. Martindale, J. E. F. Reynolds, and Royal Pharmaceutical Society of Great Britain. Council, The extra pharmacopoeia, 31st ed. London: Royal Pharmaceutical Society, 1996, pp. xxi, 2739 p. (Martindale, Reynolds, & Royal Pharmaceutical Society of Great Britain. Council, 1996); and G. M. Wilkes, Oncology Nursing Drug Handbook 2016, 20 ed. Sudbury: Jones & Bartlett Publishers, 2016, p. 1500 p. (Wilkes, 2016), relevant portions of each are incorporated herein by reference.

The present invention is based, at least in part, on the discovery that PDGFRs are expressed in TNBC, and that the inhibition of these receptors results in effects which may benefit patients at risk of developing this disease. The present invention comprises the use of the compounds of the present invention to prevent development of TNBC in high-risk patients.

Crenolanib (4-Piperidinamine, 1-[2-[5-[(3-methyl-3-oxetanyl) methoxy]-1H-benzimidazol-1-yl]-8-quinolinyl]) and its pharmaceutically acceptable salts, including but not limited to: Crenolanib Besylate, Crenolanib Phosphate, Crenolanib Lactate, Crenolanib Hydrochloride, Crenolanib Citrate, Crenolanib Acetate, Crenolanib Toluenesulphonate and Crenolanib Succinate, but may also be made available free of salts. Preparation of the compounds of the present invention. General synthetic methods for preparing the compounds of Formula I are provided in, e.g., U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), US Patent Application Publication No. US 2005/0124599 (Pfizer, Inc.) and U.S. Pat. No. 7,183,414 (Pfizer, Inc.), relevant portions incorporated herein by reference.

By way of example, crenolanib besylate is an orally bioavailable, selective, and potent type I tyrosine kinase inhibitor (TKI) of class III receptor tyrosine kinases (RTKs). The compound has the ability to inhibit both PDGFRα and PDGFRβ. Crenolanib does not inhibit any other known RTKs (e.g., VEGFR or fibroblast growth factor receptor) or serine/threonine kinases (e.g. Abl, Raf) at concentrations that are used clinically. Crenolanib has the formula:

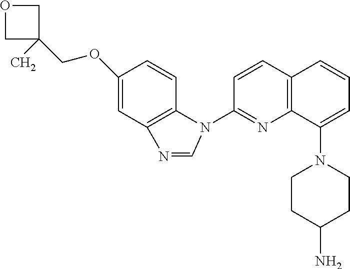 [SMILES: CC1(COc2ccc3c(c2)ncn3-c2ccc3cccc(N4CCC(N)CC4)c3n2)COC1]

In one embodiment, the present invention provides a method for preventing the development of triple negative breast cancer (TNBC) in a subject at risk by inhibiting PDGFR signaling. This comprises administering to a subject at risk of developing TNBC the compound of the present invention. Administration of the therapeutic agent can occur upon determination that the patient is considered at risk of developing TNBC.

In one aspect of this invention, the crenolanib or salt thereof may be administered to a subject systemically, for example, orally, intravenously, subcutaneously, intramuscular, intradermal, or parenterally. The compound of the present invention can also be administered to a subject locally.

The compound of the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions, and suspensions.

The daily dosage of the compound of the present invention may be varied over a wide range from 50 to 500 mg per adult human per day. For oral administration, the compositions are preferably provided in the form of tablets containing 20 to 100 milligrams. The compound of the present invention may be administered on a regimen up to three times or more per day. Optimal doses to be administered may be determined by those skilled in the art and will vary with the compound of the present invention used, the mode of administration, the time of administration, the strength of the preparation, and the details of the disease condition. Factors associated with patient characteristics, such as age, weight, and diet will call for dosage adjustments.

Pharmaceutically acceptable salts such as hydrochloride, phosphate, and lactate are prepared in a manner similar to the benzenesulfonate salt and are well known to those of moderate skill in the art. The following representative compounds of the present invention are for exemplary purposes only and are in no way meant to limit the invention, including crenolanib as crenolanib besylate, crenolanib phosphate, crenolanib lactate, crenolanib hydrochloride, crenolanib citrate, crenolanib acetate, crenolanib toluenesulphonate, and crenolanib succinate.

Definition of patients considered at risk for developing TNBC.

As used herein, patients considered at risk for developing breast cancer are as defined by the U.S. Preventive Services Task Force (USPSTF). The USPSTF considered women with a personal or family history of breast, ovarian, tubal, or peritoneal cancer or an ancestry associated with BRCA1/2 gene mutation to be at risk of developing breast cancer, and recommends that primary care clinicians assess these women and provided genetic counseling and testing (Force et al., 2019).

While there are some preventive medications available for subjects at risk of developing hormone receptor positive breast cancer, including selective estrogen receptor modulates such as tamoxifen and aromatase inhibitors/inactivators such as anastrozole or exemestane, there are no such medications available for patients at risk for TNBC (Moyer & Force, 2013). Instead, for many subjects at high risk for developing TNBC, such as those with BRCA1/2 mutations, preventive mastectomies are considered (Carbine, Lostumbo, Wallace, & Ko, 2018). However, preventive mastectomies are not a guaranteed prevention, as breast tissue can be found in the chest well, near the collarbone, and even in the abdomen, making removing all breast tissue impossible for surgeons. In addition, preventive mastectomy is associated with significant adverse physical, psychological, and emotional effects.

Thus, a need remains for a medication that prevents the development of breast cancer in patients at high risk.

Prevention of breast cancer development in subjects at risk.

Previous studies of both the present invention have either focused on the effects on the cancer cell growth in vitro or on angiogenesis. For example, Joglekar-Javadekar et al have previously shown that crenolanib inhibits the growth of inflammatory breast cancer cells (Joglekar-Javadekar et al., 2017). The proposed mechanism of action for the present invention, the prevention of TNBC in subjects considered at risk is novel.

In the MMTV-PyMT mouse model of breast cancer, the Polyoma Virus middle T antigen is expressed under the control of the mouse mammary tumor virus promoter, which results in spontaneous formation of mammary tumors in female mice. This model effectively recapitulates all of the stages of human disease, and mice develop tumors begin developing palpable mammary tumors at approximately 13 weeks of age (Guy, Cardiff, & Muller, 1992). Therefore, the MMTV-PyMT model is a highly reproducible and highly faithful model for the development of TNBC.

Animal Studies. All animal procedures were approved by the Institutional Animal Care and Use Committee at Southern University of Science and Technology. Twelve 8-wk-old female MMTV-PyMT mice were divided into six groups. The two mice in each group were littermates. Each group of mice was treated with crenolanib (20 mg/kg, dissolved in vehicle; Selleck) or vehicle [5% (vol/vol) glycerol formal; Sigma] intraperitoneally once daily for 35 d. Once palpable, tumor sizes were measured with a digital caliper every 5 d, and the volumes were calculated using the formula (length×width)/2. Data were reported as means±SE. At the end of crenolanib treatment, mice were killed, and tumors were harvested and weighted.

SDS/PAGE and Western Blot Analysis. The labeling performance of probe 1 to various bait proteins was characterized by high-resolution SDS/PAGE using the PROTEAN II xi Cell electrophoresis instrument (gel size, 16×20 cm; Bio-Rad) and Coomassie brilliant blue staining. Equal amounts of protein were separated by SDS/PAGE and transferred to membranes. Membranes were blocked in 5% (wt/vol) BSA and then incubated with a primary antibody overnight at 4° C., followed by an incubation with an HRP-conjugated anti-rabbit or anti-mouse secondary antibody (Cell Signaling Technology). Proteins were visualized using a Western ECL Substrate Kit (Bio-Rad). The primary antibodies used in this study were streptavidin-HRP (Thermo Fisher), anti-4G10 (Merck Millipore), anti-His (Cell Signaling Technology), anti-EGFR (Cell Signaling Technology), anti-ERBB2 (Cell Signaling Technology), anti-PDGFRB (Cell Signaling Technology), anti-p-STAT3 (Cell Signaling Technology), anti-p-AKT (Cell Signaling Technology), anti-AKT (Cell Signaling Technology), anti-p-ERK 1/2 (Cell Signaling Technology), anti-ERK 1/2 (Cell Signaling Technology), and anti-β-actin (Beyotime).

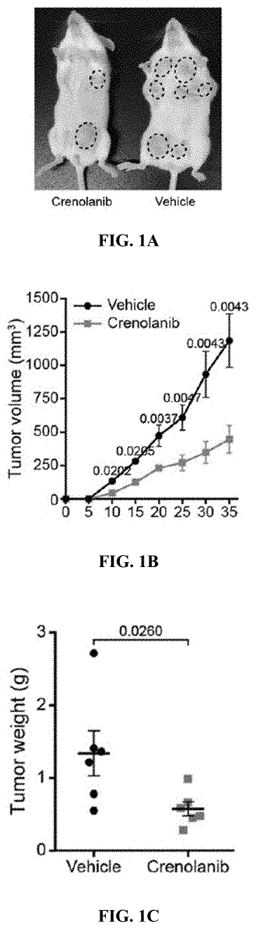

The data showed that PDGFRB abundantly expressed in a breast fibroblast cell line (human mammary fibroblasts; HMFs) but not in malignant breast cells or normal breast cells (MCF 10A) (data not shown). In vivo analysis also validated that PDGFRB was prominently expressed in tumor associated stromal cells, including spindle-shaped fibroblasts, but not in tumor tissues by using the polyoma middle T (PyMT) oncogene-driven breast cancer mouse model, which mimics all identifiable stages of human breast cancer progression (28) (data not shown). More importantly, these data show that high stromal PDGFRB expression is positively associated with breast cancer progression (data not shown), implying a strong scientific rationale for targeting the PDGFRB-associated fibroblasts in anti-breast cancer therapy. To further analyze the paracrine effects of breast cancer cells on fibroblast expansion, the inventors assessed the ligand secretion by breast cancer cells (data not shown). Results showed that high concentrations of the PDGFRB ligands PDGFB and/or PDGFC were secreted by breast cancer cells into the conditioned media (29-31) (data not shown), which in turn acted on fibroblasts to activate known PDGFR downstream targets such as AKT and ERK but not STAT3 (data not shown). To test the potential of PDGFR as a tumor therapeutic target in vivo, the inventors administered crenolanib, a highly selective inhibitor of PDGFRB (32) (Dataset S11), to 8-wk-old MMTV-PyMT mice to block stromal PDGFRB signaling. Tumor volumes in crenolanib-treated and untreated mice were measured every 5 d after dosing initiation. Beginning at day 10 after treatment, tumors derived from crenolanib-treated mice grew significantly more slowly than those derived from vehicle-treated mice, as evaluated by standard external calipers (FIGS. 1A and 1B). In addition, tumor weight per mouse at sacrifice (12 wk) was 55% lower in crenolanib-treated mice than in vehicle-treated mice (FIG. 1C). Taken together, these results show a novel therapeutic approach targeting stromal PDGFRB signalling to benefit patients with different breast cancer subtypes.

Using this model, treatment with the present invention starting at 8 weeks of age, before the development of palpable mammary tumors, significantly prevented tumor growth, and resulted in reduced the number of tumors, tumor volume, and tumor weight, as shown in FIGS. 1A, 1B, and 1C.

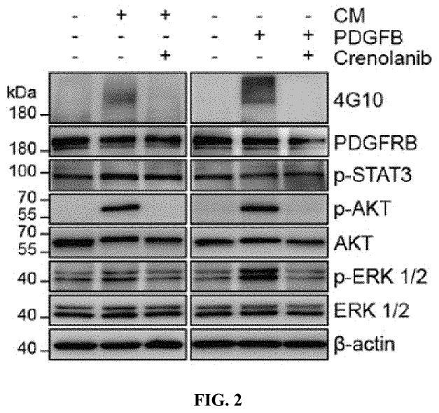

In addition, the ability of the present invention to block PDGFR signaling in a conditioned media in vitro system was investigated. In MMTV-PyMT mice, PDGFR expression in the breast tissue increases over time as the mice age and develop tumors, indicating that dysregulated PDGFR signaling may be involved in cancer development in this model. The human TNBC cell line MDA-MB-468 expresses the ligand for PDGFRβ, and when conditioned media from these cells is applied to human mammary fibroblasts, there is a marked increase in downstream pathway signaling (i.e. phosphorylated AK and ERK 1/2), which is inhibited when the fibroblasts are pre-treated with the present invention (see FIG. 2).

Thus, the present invention effectively prevents the development of tumors in subjects genetically predisposed to the disease, possibly through its activity against PDGFR signaling.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Anderson, P. O., Knoben, J. E., & Troutman, W. G. (2002). Handbook of clinical drug data (10th ed.). New York; Toronto: McGraw-Hill Medical Pub. Division.
Carbine, N. E., Lostumbo, L., Wallace, J., & Ko, H. (2018). Risk-reducing mastectomy for the prevention of primary breast cancer. Cochrane Database Syst Rev, 4, CD002748. doi: 10.1002/14651858. CD002748.pub4
Chu, B., He, A., Tian, Y., He, W., Chen, P., Hu, J., . . . Tian, R. (2018). Photoaffinity-engineered protein scaffold for systematically exploring native phosphotyrosine signaling complexes in tumor samples. Proc Natl Acad Sci USA, 115(38), E8863-E8872. doi:10.1073/pnas.1805633115
Criscitiello, C., Gelao, L., Viale, G., Esposito, A., & Curigliano, G. (2014). Investigational platelet-derived growth factor receptor kinase inhibitors in breast cancer therapy. Expert Opin Investig Drugs, 23(5), 599-610. doi:10.1517/13543784.2014.895323
Force, U. S. P. S. T., Owens, D. K., Davidson, K. W., Krist, A. H., Barry, M. J., Cabana, M., . . . Wong, J. B. (2019). Risk Assessment, Genetic Counseling, and Genetic Testing for BRCA-Related Cancer: US Preventive Services Task Force Recommendation Statement. JAMA, 322(7), 652-665. doi:10.1001/jama.2019.10987
Frings, O., Augsten, M., Tobin, N. P., Carlson, J., Paulsson, J., Pena, C., . . . Sonnhammer, E. L. (2013). Prognostic significance in breast cancer of a gene signature capturing stromal PDGF signaling. Am J Pathol, 182(6), 2037-2047. doi:10.1016/j.ajpath.2013.02.018
Goldstein, A., Pratt, W. B., & Taylor, P. (1990). Principles of drug action: the basis of pharmacology (3rd ed.). New York: Churchill Livingstone.
Goodman, L. S., Hardman, J. G., Limbird, L. E., & Gilman, A. G. (2001). Goodman and Gilman's the pharmacological basis of therapeutics (10th ed.). New York: McGraw-Hill.
Guy, C. T., Cardiff, R. D., & Muller, W. J. (1992). Induction of mammary tumors by expression of polyomavirus middle T oncogene: a transgenic mouse model for metastatic disease. Mol Cell Biol, 12(3), 954-961. doi:10.1128/mcb.12.3.954
Hammer, A. M., Sizemore, G. M., Shukla, V. C., Avendano, A., Sizemore, S. T., Chang, J. J., . . . Ostrowski, M. C. (2017). Stromal PDGFR-alpha Activation Enhances Matrix Stiffness, Impedes Mammary Ductal Development, and Accelerates Tumor Growth. Neoplasia, 19(6), 496-508. doi:10.1016/j.neo.2017.04.004
Harbeck, N., & Gnant, M. (2017). Breast cancer. The Lancet, 389(10074), 1134-1150. doi:10.1016/s0140-6736(16)31891-8
Heldin, C. H., Lennartsson, J., & Westermark, B. (2017). Involvement of PDGF ligands and receptors in tumorigenesis. J Intern Med. doi:10.1111/joim.12690
Joglekar-Javadekar, M., Van Laere, S., Bourne, M., Moalwi, M., Finetti, P., Vermeulen, P. B., . . . van Golen, K. L. (2017). Characterization and Targeting of Platelet-Derived Growth Factor Receptor alpha (PDGFRA) in Inflammatory Breast Cancer (IBC). Neoplasia, 19(7), 564-573. doi:10.1016/j.neo.2017.03.002
Katzung, B. G. (2004). Basic & clinical pharmacology (9th ed.). New York: Lange Medical Books/McGraw Hill.
Lev, D. C., Kim, S. J., Onn, A., Stone, V., Nam, D. H., Yazici, S., . . . Price, J. E. (2005). Inhibition of platelet-derived growth factor receptor signaling restricts the growth of human breast cancer in the bone of nude mice. Clin Cancer Res, 11(1), 306-314.
Martindale, W., Reynolds, J. E. F., & Royal Pharmaceutical Society of Great Britain. Council. (1996). The extra pharmacopoeia (31st ed.). London: Royal Pharmaceutical Society.
Moyer, V. A., & Force, U. S. P. S. T. (2013). Medications to decrease the risk for breast cancer in women: recommendations from the U.S. Preventive Services Task Force recommendation statement. Ann Intern Med, 159(10), 698-708. doi:10.7326/0003-4819-159-10-201311190-00717
Paulsson, J., Ehnman, M., & Ostman, A. (2014). PDGF receptors in tumor biology: prognostic and predictive potential. Future Oncol, 10(9), 1695-1708. doi:10.2217/fon.14.83
Remington, J. P., & Gennaro, A. R. (2000). Remington: the science and practice of pharmacy (20th ed ed.). Baltimore, Md.: Lippincott Williams & Wilkins.
Wilkes, G. M. (2016). Oncology Nursing Drug Handbook 2016 (20 ed.). Sudbury: Jones & Bartlett Publishers.

1. A method for preventing the development of triple negative breast cancer in a subject predisposed to breast cancer comprising administering to the subject an effective amount of crenolanib or pharmaceutically acceptable salt thereof.

2. The method of claim 1, wherein the triple negative breast cancer is not an inflammatory breast cancer.

3. The method of claim 1, wherein the effective amount of crenolanib is from about 50 mg to 500 mg per day, 100 to 450 mg per day, 200 to 400 mg per day, 300 to 500 mg per day, 350 to 500 mg per day, or 400 to 500 mg per day.

4. The method of claim 1, wherein the effective amount of crenolanib is administered at least one of continuously, intermittently, systemically, or locally.

5. The method of claim 1, wherein the effective amount of crenolanib is administered orally, intravenously, or intraperitoneally.

6. The method of claim 1, wherein the effective amount of crenolanib is administered up to three times a day for as long as the subject is at risk for development of triple negative breast cancer.

7. The method of claim 1, wherein the crenolanib is crenolanib besylate, crenolanib phosphate, crenolanib lactate, crenolanib hydrochloride, crenolanib citrate, crenolanib acetate, crenolanib toluenesulphonate, or crenolanib succinate.

8. A method for preventing the development of triple negative breast cancer in a subject comprising:
identifying a subject that is predisposed to a breast cancer; and
administering to the subject an effective amount of crenolanib or pharmaceutically acceptable salt thereof.

9. The method of claim 8, wherein the triple negative breast cancer is not an inflammatory breast cancer.

10. The method of claim 8, wherein the effective amount of crenolanib is from about 50 mg to 500 mg per day, 100 to 450 mg per day, 200 to 400 mg per day, 300 to 500 mg per day, 350 to 500 mg per day, or 400 to 500 mg per day.

11. The method of claim 8, wherein the effective amount of crenolanib is administered at least one of continuously, intermittently, systemically, or locally.

12. The method of claim 8, wherein the effective amount of crenolanib is administered orally, intravenously, or intraperitoneally.

13. The method of claim 8, wherein the effective amount of crenolanib is administered up to three times a day for as long as the subject is at risk for development of triple negative breast cancer.

14. The method of claim 8, wherein the crenolanib is crenolanib besylate, crenolanib phosphate, crenolanib lactate, crenolanib hydrochloride, crenolanib citrate, crenolanib acetate, crenolanib toluenesulphonate, or crenolanib succinate.
